(19) United States
(12) Patent Application Publication
(10) Pub. No.: US 2024/0050044 A1
(43) Pub. Date: Feb. 15, 2024

(54) MODULAR CONFIGURATION OF PATIENT MONITORS

(71) Applicant: DRAEGER MEDICAL SYSTEMS, INC., ANDOVER, MA (US)

(72) Inventor: Clifford M. RISHER-KELLY, Wells, ME (US)

(21) Appl. No.: 18/271,428

(22) PCT Filed: Dec. 16, 2021
(86) PCT No.: PCT/EP2021/086277

§ 371 Date: Jul. 7, 2023

Related U.S. Application Data
(60) Provisional application No. 63/128,454, filed on Dec. 21, 2020.

(51) Int. Cl. A61B 5/00; G09G 5/00; G06F 3/14

(52) U.S. Cl. CPC A61B 5/7445; G09G 5/006; G06F 3/1446; A61B 2562/227; A61B 2560/0443; G09G 2300/026; G06F 3/1431; G09G 2380/08

(57) ABSTRACT

A monitor system includes a first monitor configured to receive physiological sensor data from at least one physiological sensor attached to a patient, the first monitor including a first display, a first multi-contact electrical interface, and a second multi-contact electrical interface, wherein the first the second multi-contact electrical interfaces are configured to provide data signals derived from the physiological sensor data and power signals provided by a power source; and a second monitor including a second display, a third multi-contact electrical interface configured to electrically couple with the first multi-contact electrical interface, and a fourth multi-contact electrical interface configured to electrically couple with the second multi-contact electrical interface, wherein the third and the fourth multi-contact electrical interfaces are configured to receive the data signals and the power signals from the first monitor, and wherein the second display is configured to display images corresponding to the data signals.

### RELATED APPLICATIONS

This application claims the priority of U.S. Application Ser. No. 63/128,454, filed Dec. 21, 2020, the content of which is hereby incorporated by reference herein for all purposes, including the right for priority, as if set forth verbatim herein.

### TECHNICAL FIELD

The present disclosure generally relates to a patient monitor system having a mount, a smaller monitor, and a larger monitor that is adapted to removably receive the smaller monitor, with the smaller monitor and/or the larger monitor configured to be removably attached to the mount alone or in combination, and to provide for data and power transfer between the monitors and the mount.

### DESCRIPTION OF THE RELATED ART

Monitors that include electronic visual displays are utilized in a large number of applications within a wide variety of industries including, for example, the healthcare industry, the military, and the oil and gas industry. Many of the applications within such industries require such monitors to, at times, be portable, and, at other times, be stationary. For example, in the healthcare industry, when not being used in transport of a patient or when a patient is ambulatory, monitors can be connected to a monitor mount. Such monitor mounts can provide a variety of functions including providing physical support, a power source, and a conduit to one or more computer networks.

One type of monitor is a patient monitor, which is used by healthcare facilities to monitor and display information about a patient, such as vital signs, status of connected devices (e.g., physiological sensors, etc.), and the like. Patient monitors may be portable devices that travel with the patient in order to provide continuous monitoring during care. When a patient arrives at a hospital room or other treatment location, the patient monitor is often plugged into or otherwise connected to a patient monitor mount. Patient monitor mounts may provide a physical interface for the patient monitor and may be fixed to the treatment location. Patient monitor mounts may also provide electrical connection to other devices or infrastructure, such as power to recharge patient monitor batteries, network connectivity to other medical devices or hospital computer systems, and the like.

During the course of providing healthcare to patients, practitioners may connect at least one type of sensor to a patient to sense, derive, or otherwise monitor at least one type of patient medical parameter. Such patient connected sensors may be connected to a monitor that includes all relevant electronic components that enable conversion, manipulation and processing of the data sensed by the at least one type of sensor in order to generate patient medical parameters. These patient medical parameters may be stored in one or more modules and are usable by healthcare practitioners (e.g., nurses, doctors, physician assistants, or any other person charged with providing a healthcare service to a patient) in monitoring a patient and determining a course of healthcare to be provided to the patient. Additionally, or alternatively, the one or more modules may contain data, such as patient treatment data, to be transferred to the monitor mount and/or the monitor.

A monitor may be selectively connected to a patient at any point during which a healthcare professional comes into contact with the patient and may remain connected with the patient as the patient moves through various locations within a particular healthcare enterprise (e.g., hospital) or between different healthcare enterprises (e.g., an ambulance and/or different medical facilities). A monitor and/or a module can allow data representing the at least one patient medical parameter to be communicated to other systems within the healthcare enterprise. This data may then be used by different systems in further patient care.

Patient monitors may have different sizes and provide different functionalities. With current systems, each type of patient monitor typically requires a dedicated monitor mount, a dedicated controller, and a dedicated user interface. Accordingly, such monitors may not be interoperable and the performance advantages of each type of monitor may not be combined and leveraged.

In addition, there is a growing need in acute care environments to improve clinical workflow, reduce alarm fatigue, and customize medical devices to better suit hospital protocols and use models.

In view of the foregoing, there is a need for a modular system providing a universal and scalable platform including a monitor mount capable of mixed use with monitors having different sizes which are interoperable with the same controller and the same user interface, and that can be universally docked to the monitor mount. Additionally, with the objective of providing a scalable platform, there is a need to facilitate mounting a smaller monitor to a larger monitor from multiple directions to reduce the need for performing cumbersome maneuvers and to reduce cable clutter and entanglement that may impede patient care.

### SUMMARY

Embodiments described herein provide a patient monitor system a smaller monitor (first monitor) and a larger monitor (second monitor) that is adapted to removably receive the smaller monitor. The smaller monitor is further configured to provide data and power signals to the larger monitor using one or more embodiments provided herein.

One or more embodiments provide a monitor system that includes:
a first monitor configured to receive physiological sensor data from at least one physiological sensor attached to a patient, the first monitor including a first electronic visual display, a first multi-contact electrical interface, and a second multi-contact electrical interface, wherein the first the second multi-contact electrical interfaces are configured to provide data signals derived from the physiological sensor data and power signals provided by a power source; and
a second monitor including a second electronic visual display, a third multi-contact electrical interface configured to electrically couple with the first multi-contact electrical interface, and a fourth multi-contact electrical interface configured to electrically couple with the second multi-contact electrical interface, wherein the third and the fourth multi-contact electrical interfaces are configured to receive the data signals and the power signals from the first monitor, and wherein the second electronic visual display is configured to display images corresponding to the data signals.

One or more embodiments provide a monitor assembly that includes:
a first monitor configured to receive physiological sensor data from at least one physiological sensor attached to a patient, the first monitor including a housing, a first electronic visual display, and a first multi-contact electrical interface configured to transmit data signals derived from the physiological sensor data and power signals provided by a power source; and
a first monitor adapter configured to detachably couple to the first monitor to extend the housing of the first monitor, where the first monitor adapter includes a second multi-contact electrical interface configured to electrically couple to the first multi-contact electrical interface for receiving the data signals and the power signals therefrom.

### BRIEF DESCRIPTION OF THE DRAWINGS

Embodiments are described herein making reference to the appended drawings.

FIG. 1 is a logical diagram illustrating the example system including a first monitor, a second monitor, and a monitor mount.

FIGS. 2A-2F show various mounting arrangements of the modular system 200 comprising of the first monitor, the second monitor, and the monitor mount according to one or more embodiments.

FIGS. 3A and 3B are rear perspective vies of two mounting arrangements of the modular system comprising of the first monitor and the second monitor according to one or more embodiments.

FIGS. 4A-4C show perspective views of a first monitor assembly including the first monitor and an addon to the first monitor according to one or more embodiments.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded as merely examples. Accordingly, those of ordinary skill in the art will recognize that various changes and modifications of the examples described herein can be made without departing from the spirit and scope of the present disclosure. In addition, descriptions of well-known functions and constructions may be omitted for clarity and conciseness.

For example, in the description of the figures that follow, the automatic power on apparatus, system, method, and circuit are implemented in patient monitors. However, it should be understood and appreciated by one of ordinary skill in the art that the automatic power on apparatus, system, method, and circuit of the present disclosure can be implemented in other medical or electronic devices. The implementation of the automatic power on apparatus, system, method, and circuit in the patient monitors is meant only to assist in the understanding of the present disclosure and in no way is meant to limit the implementation the automatic power on apparatus, system, method, and circuit described herein.

Additionally, the terms and words used in the following description and claims are not limited to the bibliographical meanings, but are merely used to enable a clear and consistent understanding of the present disclosure. Accordingly, it should be apparent to those skilled in the art that the following description of the present disclosure is provided for illustration purposes only, and not for the purpose of limiting the present disclosure as defined by the appended claims and their equivalents.

Features from different embodiments may be combined to form further embodiments, unless specifically noted otherwise. Variations or modifications described with respect to one of the embodiments may also be applicable to other embodiments. In some instances, well-known structures and devices are shown in block diagram form rather than in detail in order to avoid obscuring the embodiments.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It is to be understood that the singular forms “a”, “an”, and “the”, include plural referents unless the context clearly dictates otherwise. Thus, for example, reference to “a processor” or “a memory” includes reference to one or more of such processors or memories.

The expressions such as “include” and “may include” which may be used in the present disclosure denote the presence of the disclosed functions, operations, and constituent elements, and do not limit the presence of one or more additional functions, operations, and constituent elements. In the present disclosure, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

In the present disclosure, the expression “and/or” includes any and all combinations of the associated listed words. For example, the expression “A and/or B” may include A, may include B, or may include both A and B.

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

The subject matter described herein is directed to systems and apparatuses directed to monitors (e.g., display monitors having visual electronic displays) and monitor mounts providing physical support and, in some cases, power and access to a communications/computer network. Use of such systems and apparatuses can, for example, occur in a medical environment such as the scene of a medical event, an ambulance, a hospital or a doctor's office. When a patient undergoes initial patient monitoring in such an environment, a minimum set of sensors can be connected to a patient to collect various types of patient information (e.g., physiological information) as described in detail herein. As a patient is moved from one area of care within the medical environment to another area of care, the patient monitor can travel with the patient. In some situations, the patient monitor can be mounted to a monitor mount to provide for stationary observation of the patient information on a visual electronic display.

During the course of patient monitoring, the number of sensors can also increase due to increased testing and/or monitoring of the patient. In such a scenario, a patient monitor initially monitoring the patient can be docked onto a monitor mount having a second, larger monitor in order to expand the number of sensors available for patient monitoring and/or increase the number of patient parameters on a single visual electronic display by docking the smaller patient monitor to or within a larger patient monitor. The initial patient monitor can either remain within the larger patient monitor or be removed from the larger patient monitor.

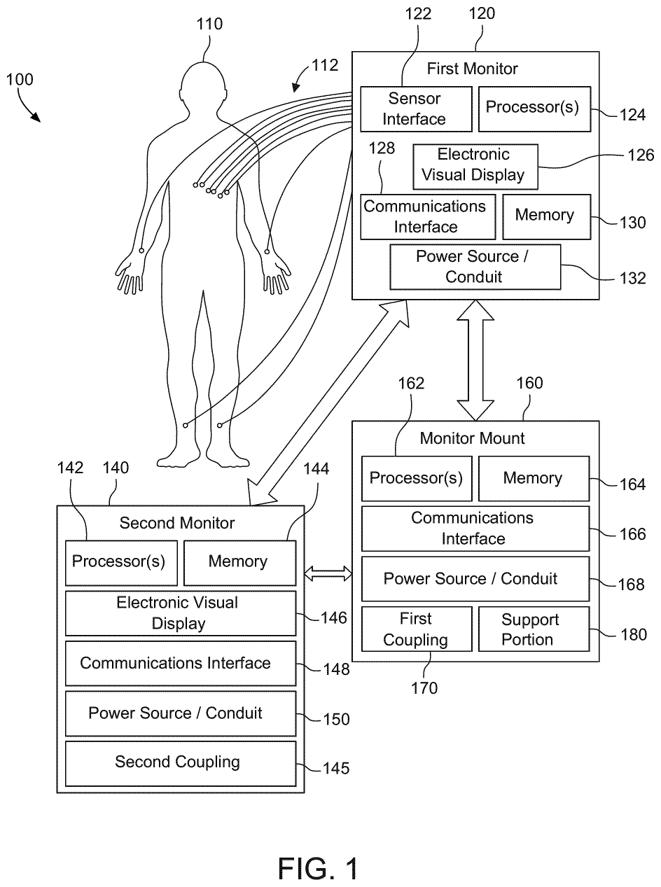

FIG. 1 is a logical diagram illustrating an example system 100 including a first monitor 120, a second monitor 140, and a monitor mount 160. In an exemplary implementation, monitor mount 160 may be detachably secured to a support structure (not shown) (e.g., a wall-mounted arm) via any attachment mechanism (not shown) such as a Video Electronics Standards Association (VESA) mounting interface adapted to an attachment mechanism in a hospital room in which a patient 110 is being monitored and/or treated via one or more modules, for example one or more physiological sensors and/or medical devices.

Monitor mount 160 of system 100 may detachably secure second monitor 140, and second monitor 140 may detachably secure first monitor 120. First monitor 120 may be initially mounted or otherwise detachably secured to the second monitor 140 to form a two-monitor system 100, and two-monitor system 100 may subsequently be detachably secured to monitor mount 160. Alternatively, second monitor 140 may be first mounted or otherwise detachably secured to monitor mount 160, and first monitor 120 may be subsequently inserted into or otherwise mounted within a mounting area defined by second monitor 140, monitor mount 160, or both second monitor 140 and monitor mount 160. For example, the mounting area may be defined by a volume or cavity formed when second monitor 140 and the monitor mount 160 are coupled together.

Therefore, example system 100 provides an interconnected, versatile, and comprehensive patient care solution with a high degree of configurability. Example system 100 acquires data at the bedside and on transport, without having to disconnect a patient as he or she is moved from care area to care area. Example system 100 can be scaled depending on the patient's changing acuity level and medical devices can be customized to better suit hospital protocols and use models. Accordingly, example system 100 thereby improves clinical workflow.

Monitor mount 160 may detachably secure (or otherwise physically interface with) both of first monitor 120 and second monitor 140, alone or in combination. As will be described in further detail below, first monitor 120 has a shape and size which differs from that of second monitor 140. Nonetheless, both of first monitor 120 and second monitor 140 are able to be concurrently secured to monitor mount 160.

First monitor 120 can, for example, be a patient monitor that is used to monitor various physiological parameters for patient 110. With such a variation, first monitor 120 can include a sensor interface 122 that can be used to connect via wired and/or wireless interfaces to one or more physiological sensors and/or medical devices 112 (e.g., electrocardiogram (ECG) electrodes, oxygen saturation (SpO2) sensor, non-invasive blood pressure (NIBP), blood pressure cuffs, apnea detection sensors, end-tidal carbon dioxide (etCO2), respirators, temperature, and other similar physiological data.) associated with patient 110. First monitor 120 can include one or more processors 124 (e.g., programmable data processors, etc.) which can execute various instructions stored in memory 130 of first monitor 120. Various data and graphical user interfaces can be conveyed to a user via an electronic visual display 126 included in first monitor 120. This information can, for example, relate to the measured physiological parameters of patient 110 and the like (e.g., ECG waveforms, blood pressure, heart related information, pulse oximetry, respiration information, temperature, etc.). Other types of information can also be conveyed by electronic visual display 126.

In examples, first monitor may include a video processor for generating information and images to be displayed on electronic visual display 126. In some variations, the electronic visual display 126 includes a touch screen interface that allows a user of first monitor 120 to input data and/or modify the operation of first monitor 120.

First monitor 120 can additionally include a communications interface 128 which allows first monitor 120 to directly or indirectly (via, for example, monitor mount 160) access one or more computing networks. Communications interface 128 may include various network cards/interfaces to enable wired and wireless communications with such computing networks. Communications interface 128 may also enable direct (i.e., device-to-device, etc.) communications (i.e., messaging, signal exchange, etc.) such as from monitor mount 160 to first monitor 120.

First monitor 120 can optionally also include a power source and/or conduit 132 that may be used to power the various components of first monitor 120 (and optionally various components of second monitor 140 and/or monitor mount 160). Power source/conduit 132 may include a self-contained power source such as a battery and/or power source/conduit 132 may include an interface to be powered through an electrical outlet (either directly or indirectly by way of second monitor 140 and/or monitor mount 160). In some variations, first monitor 120 can only be powered when secured or otherwise connected to one or more of second monitor 140 and monitor mount 160. However, first monitor 120 may have its own battery to operate the first monitor without being connected to second monitor 140 or monitor mount 160. This battery may be removable and may be recharged by an external power source (e.g., when connected to monitor mount 160).

Second monitor 140 may include one or more processors 142 (e.g., programmable data processors, etc.) which can execute various instructions stored in memory 144 of second monitor 140. Various data and graphical user interfaces can be conveyed to a user via an electronic visual display 146 included in second monitor 140. This information may, for example, relate to the measured physiological parameters of patient 110 and the like (e.g., blood pressure, heart related information, pulse oximetry, respiration information, thermoregulation, neonatal information, ventilator information, anesthesia information, incubation information, etc.) as received from first monitor 120. Other types of information can also be conveyed by electronic visual display 146. I

In examples, first monitor may include a video processor for generating information and images to be displayed on electronic visual display 126. In some variations, the electronic visual display 126 includes a touch screen interface that allows a user of first monitor 120 to input data and/or modify the operation of first monitor 120.

Second monitor 140 may additionally include a communications interface 148 which allows second monitor 140 to directly or indirectly (via, for example, the first monitor 120 and/or monitor mount 160) access one or more computing networks. Communications interface 148 may include various network cards/interfaces to enable wired and wireless communications with such computing networks. Communications interface 148 may also enable direct (i.e., device-to-device, etc.) communications (i.e., messaging, signal exchange, etc.) such as from monitor mount 160 to second monitor 140 and first monitor 120 to second monitor 140.

Second monitor 140 may optionally also include a power source and/or conduit 150 that can be used to power various components of second monitor 140 (and optionally various components of first monitor 120). Power source/conduit 150 may include a self-contained power source such as a battery and/or power source/conduit 150 may include an interface to be powered through an electrical outlet (either directly or by way of first monitor 120 and/or monitor mount 160). In some variations, second monitor 140 may only be powered and render information when secured or otherwise connected to one or more of first monitor 120 and monitor mount 160.

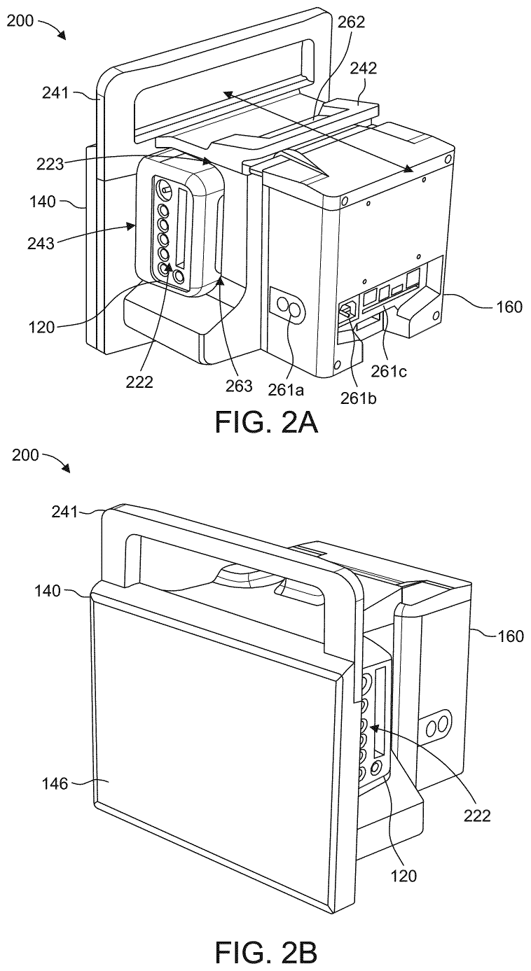

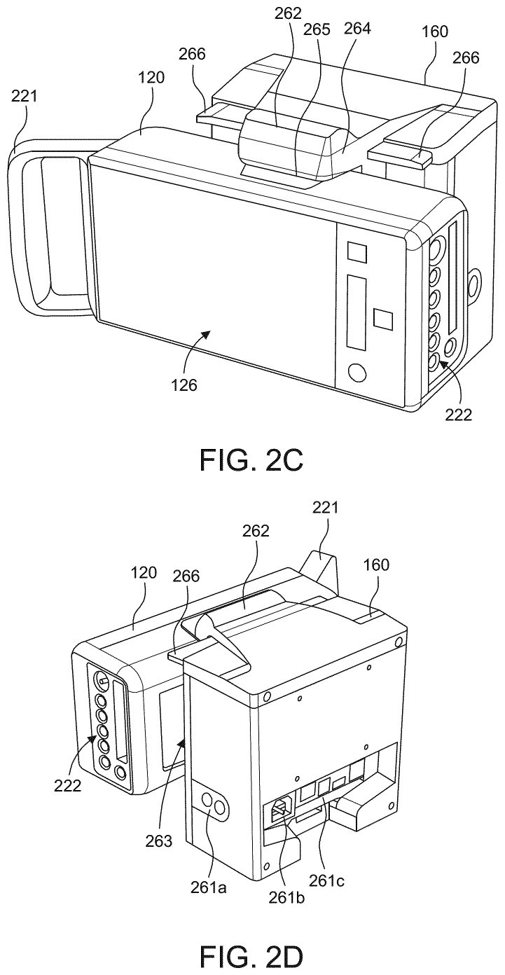

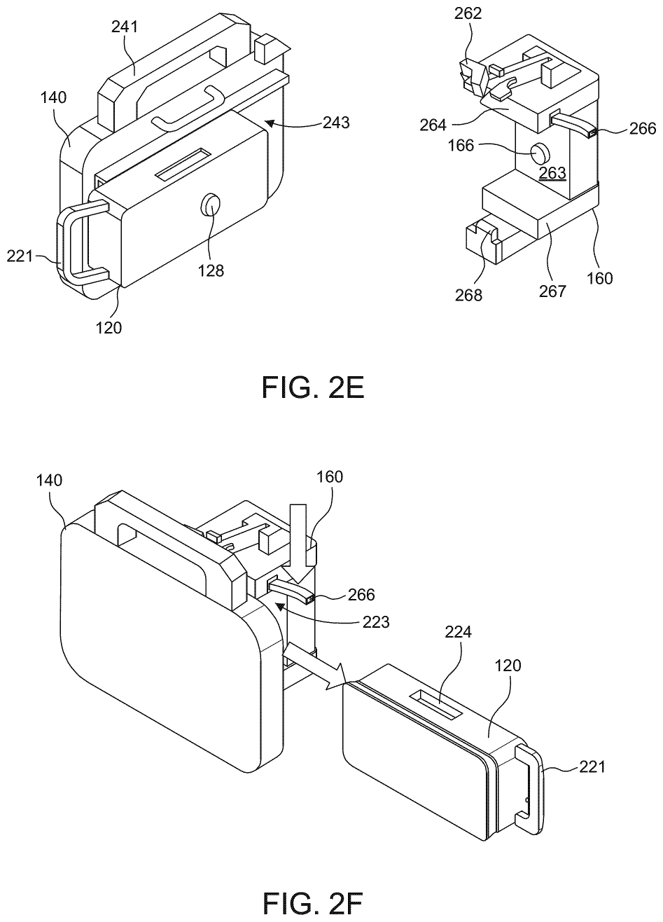

Second monitor 140 may include a second coupling 145 which is configured to detachably secure first monitor 120. In some variations, second coupling 145 may be positioned in a cavity of second monitor 140, as herein described with reference to FIG. 2A. The cavity may be defined in a lateral direction of the second monitor 140 and have open side portions for receiving first monitor 120. For example, the user can visually confirm the location of second coupling 145 and transversely insert first monitor 120 into second monitor 140. In some variations, the cavity may have an open top portion instead of open side portions such that first monitor 120 can be dropped into second monitor 140 from above; and removed (e.g., lifted out) from second monitor 140 from above.

Monitor mount 160 may include one or more processors 162 (e.g., programmable data processors, etc.) which may execute various instructions stored in memory 164 of monitor mount 160. Monitor mount 160 may additionally include a communications interface 166 which allows monitor mount 160 to directly or indirectly access one or more computing networks. Communications interface 166 may include various network cards/interfaces to enable wired and wireless communications with such computing networks. Communications interface 166 may also enable direct (i.e., device-to-device, etc.) communications (i.e., messaging, signal exchange, etc.) such as with first monitor 120 and/or second monitor 140.

Monitor mount 160 may optionally also include a power source and/or conduit 168 that can be used to power the various components of monitor mount 160 and/or first monitor 120 and/or second monitor 140 when secured to monitor mount 160. Power source/conduit 168 may include a self-contained power source such as a battery and/or power source/conduit 168 may include an interface to be powered through an electrical outlet.

Any of processors 124, 142, 162 may acquire data from any of the monitor mount 160 and one or more of monitors 120, 140 and store the acquired data in a memory and, upon connection of monitor mount 160 and one or more of monitors 120, 140, transfer the data stored in the memory to monitor mount 160 or one or more of the monitors 120, 140. The data may include any of patient identification data including information identifying a patient; patient parameter data representing at least one type of patient parameter being monitored; and device configuration data including information associated with configuration settings for monitor mount 160 and/or the one or more monitors 120, 140.

Monitor mount 160 may optionally also include any mounting interface, such as a VESA mounting interface for mounting the monitor mount at the bedside, from the ceiling, on a wall of the room, or even outside the room for isolation purposes.

Monitor mount 160 may optionally also include an interface configured to receive a connector of a cable or wired connection for connecting a module, a monitor, other external unit or the like.

Monitor mount 160 can optionally also include one or more recesses for facilitating removal of first monitor 120 and/or second monitor 140.

In some variations, the one or more processors 162 and memory 164 are omitted such that monitor mount 160 provides only physical support and optionally a power source.

Monitor mount 160 may have a shape and size which allows monitor mount 160 to detachably secure both of first monitor 120 and second monitor 140 such that respective monitors 120 and 140 may be removed by a user when desired.

Monitor mount 160 may include a first coupling 170 to allow first monitor 120 and/or second monitor 140 to be secured to monitor mount 160. Monitor mount 160 may secure each of first monitor 120 and second monitor 140 individually or both of first monitor 120 and second monitor 140 concurrently. In other words, first coupling 170 may be configured to accept either first monitor 120 or second monitor 140 such that monitor mount 160 is configured to mount first monitor 120 alone, second monitor 140 alone, or a combination of first monitor 120 and second monitor 140. First coupling 170 may include any mechanical attachment means such as a ledge, a rail, a rib, an abutment, a cleat, and the like, or any combination thereof.

First coupling 170 may additionally or alternatively include different securing mechanisms including magnetic and/or electromagnetic locking mechanisms which cause first monitor 120 to selectively be secured to monitor mount 160. In some cases, first monitor 120 may slide into and out of first coupling 170 from one or more lateral directions (i.e., from one or more sides of the monitor mount 160) while in other variations, first monitor 120 can be mounted to and removed from the front face the monitor mount 160. In some implementations, first monitor 120 can both slide into and out of first coupling 170 from one or more lateral directions and be mounted to and removed from the front face of monitor mount 160.

The positioning of first monitor 120 when secured to monitor mount 160 may be such that communications interface 128 on first monitor 120 aligns with communications interface 166 of monitor mount 160 to allow, for example, a direct connection (e.g., electrical connection). In other variations, communications interface 128 of first monitor 120 may exchange data with communications interface 166 of monitor mount 160 wirelessly (via, for example, optical communication by way of respective optical windows on first monitor 120 and monitor mount 160). For example, both communication interfaces 128 and 166 may include bi-directional phototransceivers that are configured for bi-directional communication. Communications interface 128 of first monitor 120 may be located on a back facing portion of first monitor 120, whereas communications interface 166 may be located on a front facing portion of monitor mount 160 so that the back facing portion of first monitor 120 and the front facing portion of monitor mount 160 face each other when first monitor 120 is mounted to the monitor mount 160.

The positioning of first monitor 120 when secured to monitor mount 160 may also align power source/conduit 132 of first monitor 120 to be coupled to the power source/conduit 168 of monitor mount 160, enabling monitor mount 160 to power first monitor 120.

Monitor mount 160 may include a support portion 180 to allow second monitor 140 to be secured to monitor mount 160. Support portion 180 may be positioned at a top of monitor mount 160 or a bottom of monitor mount 160. Support portion 180 may include any mechanical attachment means such as a ledge, a rail, a rib, an abutment, a cleat, and the like, or any combination thereof. The positioning of second monitor 140 when secured to monitor mount 160 may be such that communications interface 148 on second monitor 140 aligns with communications interface 166 of monitor mount 160 to allow, for example, a direct connection (e.g., electrical connection). In other variations, communications interface 148 of second monitor 140 may exchange data with communications interface 166 of monitor mount 160 wirelessly (via, for example, optical communication by way of respective optical windows on second monitor 140 and monitor mount 160). For example, both communication interfaces 128 and 166 may include bi-directional phototransceivers that are configured for bi-directional communication.

Communications interface 148 of second monitor 140 may be located on a back portion of second monitor 140. Communications interface 148 of second monitor 140 may be located on a back facing portion of second monitor 140, whereas communications interface 166 may be located on a front facing portion of monitor mount 160 so that the back facing portion of second monitor 140 and the front facing portion of monitor mount 160 face each other when second monitor 140 is mounted to monitor mount 160. As used herein, the terms “front facing portion” and “front side” are intended to refer to a portion or side of an element which faces a user of system 100, such as electronic visual display 126 of first monitor 120 or electronic visual display 146 of second monitor 140, while the terms “back facing portion” and “rear side” are intended to refer to a portion or side of an element which faces away from a user of system 100.

The support portion 180 can enable front-to-back docking of the second monitor 140 to the monitor mount 160 by providing a shelf or similar feature extending outwardly. This feature of the support portion 180 can support and/or disperse the weight of the second monitor 140 during positioning of the second monitor 140. For example, a user attempting to position the second monitor 140 onto the monitor mount 160 can rest the second monitor 140 on the support portion 180 during the positioning while attaching the back portion of the second monitor 140 to the first coupling 170. The support portion 180 can support a bottom face of the second monitor 140.

Alternatively, or additionally, support portion 180 may enable hanging or suspension of a handle of second monitor 140 from monitor mount 160 by providing any mechanical attachment means such as a ledge, a rail, a rib, an abutment, a cleat, and the like, or any combination thereof extending laterally from the top portion of monitor mount 160. This feature of support portion 180 may support and/or disperse the weight of second monitor 140 during positioning of second monitor 140. For example, a user attempting to position second monitor 140 to monitor mount 160 may hang or suspend a handle of second monitor 140 from support portion 180 during positioning while attaching the back portion of second monitor 140 to first coupling 170.

The positioning of second monitor 140 when secured to monitor mount 160 may also align power source/conduit 150 of second monitor 140 so as to be coupled to power source/conduit 168 of monitor mount 160, enabling monitor mount 160 to power second monitor 140 or vice-versa. In some variations, the positioning of second monitor 140 when secured to monitor mount 160 and/or when first monitor 120 is also secured to the monitor mount 160, this arrangement may also align power source/conduit 150 of second monitor 140 to be coupled to power source/conduit 132 of first monitor 120 (which in turn is connected to power source/conduit 168 of monitor mount 160), enabling first monitor 120 to power second monitor 140.

The modular mounting of the three devices 120, 140, and 160 will now be described in greater detail. The modular mounting may allow first monitor 120 to dock into monitor mount 160 from the front surface of monitor mount 160, allow first monitor 120 to dock into monitor mount 160 by sliding first monitor 120 in from the left and/or the right lateral side of monitor mount 160, allow the combination of first monitor 120 and second monitor 140 to dock to monitor mount 160, allow first monitor 120 to slide out of the combination of monitor mount 160 and second monitor 140 while monitor mount 160 and second monitor 140 remain mechanically coupled to one another, allow second monitor 140 to be mounted to monitor mount 160 in the absence of the first monitor mount 120, and any combination thereof.

FIGS. 2A-2F show various mounting arrangements of modular system 200 comprising of first monitor 120, second monitor 140, and monitor mount 160 according to one or more embodiments.

FIG. 2A is a rear perspective view of all three devices 120, 140, and 160 coupled together. FIG. 2B is a front perspective view of all three devices 120, 140, and 160 coupled together. Second monitor 140 may include electronic visual display 146 at its front side and a handle 241 at its top side to facilitate user handling of the unit. It will be appreciated that the placement of handle 241 may vary and two or more handles may also be provided. Second monitor 140 may further include a coupling mechanism 242 such as a cleat that extends from handle 241 and is configured to mechanically engage with and couple to monitor mount 160. Monitor mount 160 may include a corresponding coupling mechanism 262 such as a latch that mechanically engages with coupling mechanism 242. The two coupling mechanisms 242 and 262 may be mechanically interlocked with each other by maneuvering at least one of devices 140 and 160 in the direction of the arrow such that the two coupling mechanisms 242 and 262 are brought into contact.

Second monitor 140 may have a rear mounting area 243 at which first monitor 120 may be received and mounted. Similarly, monitor mount 160 may have a front mounting area 263 at which first monitor 120 may be received and mounted. When second monitor 140 is mounted to monitor mount 160, rear mounting area 243 and front mounting area 263 define a volume or a cavity 223 in which first monitor 120 can be mounted, inserted, or otherwise arranged.

Various connector ports are also shown. For example, first monitor 120 includes various connector ports 222 as part of its sensor interface 122 that are arranged on one lateral side of the first monitor 120. Monitor mount 160 may include network ports 261a for connecting to a computing network, a power port 261b for connecting to a power source, and input/output (I/O) ports 261c for connecting to external I/O devices and peripherals.

FIG. 2C is a front perspective view of first monitor 120 and monitor mount 160 coupled together without second monitor 140. FIG. 2D is a rear perspective view of first monitor 120 and monitor mount 160 coupled together without second monitor 140.

As illustrated in FIG. 2C, first monitor 120 includes the electronic visual display 126 at its front side and a handle 221 at the lateral side arranged opposite to the side at which the connector ports 222 are located. Handle 221 may facilitate handling of first monitor 120 and the mounting of first monitor 120 into the various configurations described herein. However, handle 221 is optional and may not be present to allow for a smaller footprint.

A coupling mechanism 262 of the monitor mount 160 is also shown. In this embodiment, coupling mechanism 262 is arranged on a top side of a mechanical arm 264 that extends outward from the main body of monitor mount 160. Mechanical arm 264 defines, at least in part, a front mounting area 263 into which first monitor 120 is received. The underside of mechanical arm 264 includes a coupling mechanism 265, such as a latch, that mechanically engages with and couples to a corresponding coupling mechanism, coupling structure, or coupling area (not shown) of first monitor 120. Monitor mount 160 may further include latching release paddles 226 configured to disengage coupling mechanism 265 from first monitor 120 for undocking first monitor 120 from monitor mount 160.

FIG. 2E is a rear perspective view of monitors 120 and 140 coupled together and detached from monitor mount 160. As illustrated in FIG. 2E, first monitor 120 includes its communications interface 128 at its rear side and monitor mount 160 includes communication interface 166 at its front side and arranged to be in alignment with communications interface 128 when first monitor 120 is mounted to monitor mount 160.

Monitor mount 160 may further include a mechanical arm 267 that extends from the body of monitor mount 160. Together, arms 264 and 267 may define front mounting area 263. Front mounting area 263 may be a recessed area into which first monitor 120 may be inserted for mounting to monitor mount 160. Mechanical arm 267 may further include another coupling mechanism 268 configured to engage with and couple to a bottom portion of first monitor 120.

FIG. 2F is a front perspective view of all three devices 120, 140, and 160 during an undocking maneuver or separation maneuver of first monitor 120 while second monitor 140 remains mounted to monitor mount 160. In FIG. 2F, first monitor 120 is released from coupling mechanism 265 by depressing the latching release paddle 266. Once first monitor 120 is released from coupling mechanism 265, it may by pulled out of cavity 223 in a lateral direction by using, for example, handle 221 of first monitor 120. One-handed docking and undocking of first monitor 120 may also be performed without using handle 221, which may or may not be present.

As further illustrated in FIG. 2F, a coupling area 224 of first monitor 120 can be seen. In this example, coupling area 224 is a recess configured to receive or otherwise engage with coupling mechanism 265 of monitor mount 160 to be interlocked therewith, in order to detachably secure first monitor 120 to monitor mount 160. First monitor 120 may also include a second coupling area (not illustrated) at its underside to engage with coupling mechanism 268 for detachably securing first monitor 120 to monitor mount 160.

It will also be appreciated that first monitor 120 may be inserted and removed from the other lateral (left) side of the modular assembly. For insertion on the other lateral side, first monitor 120 can be rotated 180 degrees so that handle 221 is outward facing from cavity 223. This invertible insertion of first monitor 120 into a rear mounting area 243 (or cavity 223) enables a dual-entry system that allows first monitor 120 to be mounted from either lateral side to accommodate dynamic patient environments. The of first monitor 120 side having connector ports 222 may be inserted into cavity 223 first, regardless of whether first monitor 120 is inserted from the left side or the right side. Also, electronic visual display 126 faces the rear side of second monitor 140 and communications interface 128 faces and is aligned with the communications interface 166 regardless of whether first monitor 120 is inserted from the left side or the right side. Handle 221 may be used for both insertion of first monitor 120 into cavity 223 and for removal therefrom, which can be done by one hand. The placement of the coupling areas of first monitor 120 helps ensure that communications interface 128 is aligned with communications interface 166 when coupling mechanisms 265 and 268 are engaged therewith.

In some embodiments, second monitor 140 completely surrounds first monitor 120 creating a thermal challenge for first monitor 120 to dissipate heat. In order to improve the thermal performance of first monitor 120 in dissipating heat, an internal metal plate (not illustrated) may be provided inside the housing of first monitor 120. The internal metal plate may be the same size or substantially the same size as the as electronic visual display 126 of first monitor 120 and may be disposed directly behind electronic visual display 126. The main processor of first monitor 120 may also be thermally connected to this or another internal metal plate. In addition to an internal metal plate, another metal heat-sink structure may be arranged at a rear housing of first monitor 120 to facilitate distribution of heat throughout the enclosure. Such metal structures located at the front and rear of the housing may be provided to dissipate and/or distribute heat throughout the enclosure and to reduce hot-spots and thermal overloading.

Further embodiments are directed to electrical connections between first monitor 120 and second monitor 140 to transfer data signals (e.g., video and audio signals), control signals, and power signals therebetween when first monitor 120 is docked or mounted to rear mounting area 243 of second monitor 140. Multi-contact electrical interfaces may be used to form electrical connections between monitors 120 and 140. Contacts may include electrical contact pads (e.g., metal pads), balls, pins, pin holes, and the like.

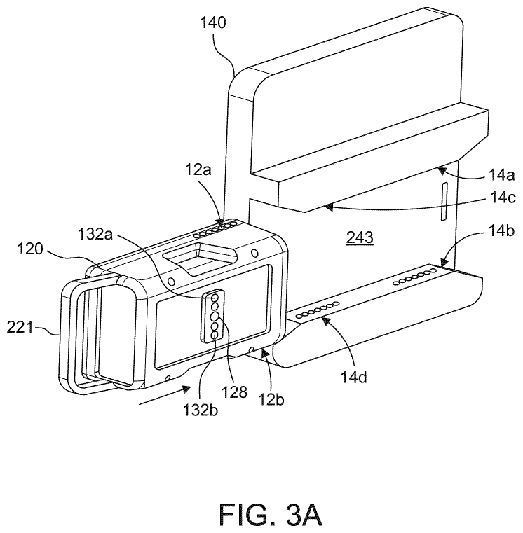

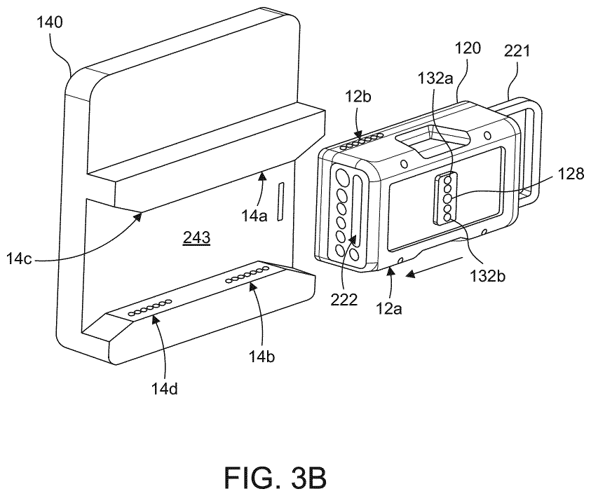

FIGS. 3A and 3B are rear perspective vies of two mounting arrangements of the modular system comprising first monitor 120 and second monitor 140 according to one or more embodiments. In particular, FIGS. 3A and 3B show a dual-entry system in which first monitor 120 is inserted into rear mounting area 243 of second monitor 140 from either a left entry point moving in the right lateral direction, as is in FIG. 3A, or a right entry point moving in the left lateral direction, as is in FIG. 3B. The orientation of first monitor 120 is rotated 180 degrees depending on the insertion direction.

First monitor 120 may include two multi-contact electrical interfaces 12a and 12b arranged to interface with a corresponding two multi-contact electrical interfaces of second monitor 140. In one example, two 7-contact connectors may be provided (14 contacts made in total) to be simultaneously connected between monitors 120 and 140 to establish the requisite electrical connections. That is, first monitor 120 may include a first 7-contact electrical interface 12a that is electrically connected to a first 7-contact electrical interface 14a of second monitor 140 to form a first connector with 7-established electrical connections. Additionally, first monitor 120 may include a second 7-contact electrical interface 12b that is electrically connected to a second 7-contact electrical interface 14b of the second monitor 140 to form a second connector with 7-established electrical connections. Both first electrical interface 12a and second electrical interface 12b may be formed simultaneously to form a total of 14 electrical connections between monitors 120 and 140 when first monitor 120 is inserted into rear mounting area 243 from a first lateral entry or insertion point.

TABLE 1 below provides one example of pin assignments according to an embodiment using 14 electrical connections for the 7-contact electrical interfaces 12a and 12b. The 7-contact electrical interfaces 14a and 14b would also match these pin arrangements. Likewise, the 7-contact electrical interfaces 14d and 14c would also match these pin arrangements.

TABLE 1

| Pin | Signal | Description |
|---|---|---|
| 1 | SYS_PWR+ | Common power rail for the first and second |
| 2 | SYS_PWR− | monitors. Each pin rated for 3 amps DC at 10 |
| | | mohm contract resistance. |
| 3 | DOUT0+ | FPDL High speed data link lane 0. |
| 4 | DOUT0− | Specifications per 4.3 |
| 5 | DOUT1+ | FPDL High speed data link lane 1. |
| 6 | DOUT1− | Specifications per 4.3 |
| 7 | Interrupt | Common interrupt bus. Single ended 50 ohm. |
| 8 | Ground | |
| 9 | USB+ | USB 2.0 differential pair. 90-ohm impedance. |
| 10 | USB− | |
| 11 | I2C SCL | I2C clock. Single ended 50 ohm. |
| 12 | I2C SDA | I2C data. Single ended 50 ohm. |
| 13 | SYS_PWR+ | Common power rail for the first and second |
| 14 | SYS_PWR− | monitors. Each pin rated for 3 amps DC at 10 |
| | | mohm contract resistance. |

Moreover, the multi-contact electrical interfaces on either the first monitor 120 or the second monitor 140 may be duplicated to enable the dual-entry feature of first monitor 120 that is accomplished by inverting first monitor 120 rotated by 180 degrees depending on its entry direction into rear mounting area 243. Thus, two 7-contact electrical interfaces may be provided on one of monitors 120 or 140 and four 7-contact electrical interfaces may be provided on the other of the monitors 140 or 120 to enable the dual-entry feature.

For example, in addition to the first two 7-contact electrical interfaces, second monitor 140 may include a third 7-contact electrical interface 14c that is electrically connected to second 7-contact electrical interface 12b of first monitor 120 to form a third connector with 7-established electrical connections. Additionally, second monitor 140 may include a fourth 7-contact electrical interface 14d that is electrically connected to first 7-contact electrical interface 12a of first monitor 120 to form a fourth connector with 7-established electrical connections. Both the third connector and the fourth connector may be formed simultaneously to form a total of 14 electrical connections between monitors 120 and 140 when first monitor 120 is inserted into rear mounting area 243 from a second lateral entry or insertion point. The second lateral entry or insertion point is located at an opposite lateral side of rear mounting area 243 or cavity 223 from the first lateral entry or insertion point.

Alternatively, in addition to the first two 7-contact electrical interfaces, first monitor 140 may include a third 7-contact electrical interface that is electrically connected to the second 7-contact electrical interface of first monitor 120 to form a third connector with 7-established electrical connections. Additionally, first monitor 120 may include a fourth 7-contact electrical interface that is electrically connected to the first 7-contact electrical interface of second monitor 140 to form a fourth connector with seven established electrical connections. Both the third connector and the fourth connector may be formed simultaneously to form a total of 14 electrical connections between monitors 120 and 140 when first monitor 120 is inserted into rear mounting area 243 from a second lateral entry or insertion point. The second lateral entry or insertion point is located at an opposite lateral side of rear mounting area 243 or cavity 223 from the first lateral entry or insertion point.

In summary, first monitor 120 may include two sets of two 7-contact electrical interfaces with only one set used at a time. Which set is used depends on the orientation of first monitor 120 in rear mounting area 243, which ultimately depends on the insertion or entry direction of first monitor 120 into rear mounting area 243. Alternatively, second monitor 140 may include two sets of two 7-contact electrical interfaces with only one set used at a time. In this example, only 14 electrical contacts are made at a time, leaving 14 contacts on first monitor 120 or 14 contacts on second monitor 140 unused.

While the contacts of each multi-contact electrical interfaces may be any type of electrical contact (e.g., contact pads, balls, pins, pin holes, and the like), the contacts of first monitor 120 may be implemented with sealed pads, allowing it to have an IPX4 rating for water resistance. Another side of the connector implemented on second monitor 140 may comprise small spring contacts that align and make contact with the sealed pads of first monitor 120.

Accordingly, links are formed between first monitor 120 and second monitor 140 based on a Mobility DisplayPort protocol, also known as MyDP, and a Universal Serial Bus protocol (USB). These two protocols may be implemented with 5 pins each, for a total of 10 pins. In addition to the two links, power and two control lines are established between monitors 120 and 140. This translates into needing two 7 pin connectors (i.e., two 7-contact electrical interfaces). Thus, two 7-contact electrical interfaces of first monitor 120 are electrically coupled to two 7-contact electrical interfaces of second monitor 140, as described above. Of course, this is merely one example configuration and the design requirements may differ or change for other types of monitors. Thus, the arrangement is not limited to using two 7-contact electrical interfaces fora total of fourteen total contacts.

In addition, second monitor 140 may comprise video multiplexors configured to bring the two connectors back to one connector for the two different functions. In other words, video multiplexers may be connected to electrical interfaces 14a-14d to perform multiplexing based on which electrical interface and which contacts are receiving video data.

To reduce the pin count, video may be encoded to a MyDP display standard. MyDP encoding and decoding may be performed with a video encoder chip, which may be used for conversion from High-Definition Multimedia Interface (HDMI) to MyDP and back again to HDMI. Audio signals may also be encoded and decoded along with the video. Alternatively, a flat panel display link III, better known as FPD-Link (FPDL) III, may be used (see e.g., TABLE 1). FDP-Link III is an interface used to transport video from point to point. This interface enables the transport of high-definition digital video, as well as a bidirectional control channel, over a low-cost cable, either twisted pair or coax. Thus, this link may be used to transmit video and audio information from the first monitor 120 to the second monitor 140 on a single pair of wires/contacts.

In addition, communications interface 128 is shown as an optical transceiver that is configured to be aligned with a corresponding optical transceiver of communications interface 166 for exchanging communications and data. First monitor 120 may further include a conduit (not separately shown) of power source/conduit 132 that includes a power contact 132a and a ground contact 132b that may be used to receive power from monitor mount 160 when first monitor 120 is mounted thereto. When first monitor 120 is mounted to monitor mount 160, power and ground contacts of the conduit of power source/conduit 132 are in electrical interface with a power contact and a ground contact of power source/conduit 168 of monitor mount 160, respectively. First monitor 120 may be configured to transfer power received from monitor mount 160 to second monitor 140 via the power contacts of the multi-contact electrical interfaces 12a and 12b.

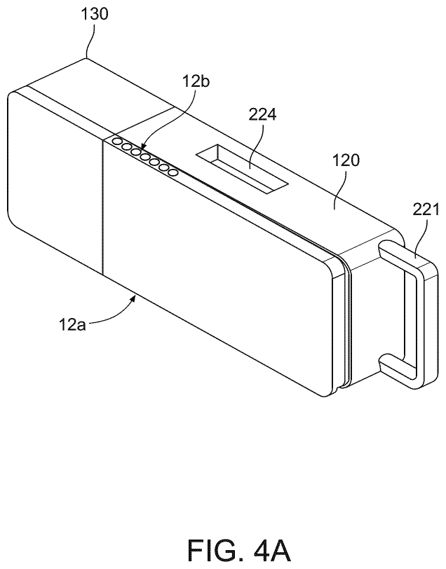

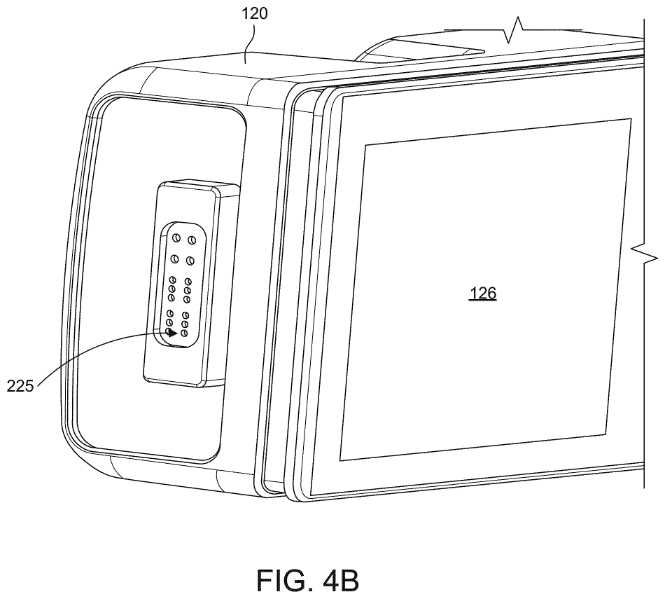

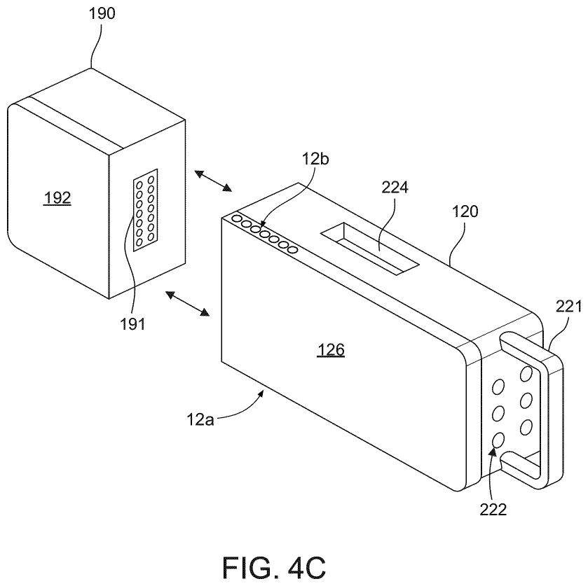

FIGS. 4A-4C show perspective views of a first monitor assembly including first monitor 120 and an add-on to first monitor 120 according to one or more embodiments. In particular, a first monitor adapter 190 is provided as an end cap that electrically and mechanically couples to a lateral side of first monitor 120. First monitor adapter 190 may be used for one or more of the following reasons: (1) to provide multi-contact electrical interfaces 12a and 12b for a first monitor 120 that does not have such contacts; (2) to increase the area of electronic visual display 126 and provide more functionality; and/or (3) to house additional electronics, including an auxiliary battery that may be used to extend the overall battery life of first monitor 120.

In FIGS. 4A-4C, first monitor adapter 190 is added on the opposite side from patient connector ports 222 to allow first monitor 120 to be docked to second monitor 140. In other words, first monitor adapter 190 may not only provide multi-contact electrical interfaces 12a and 12b for interfacing with corresponding multi-contact electrical interfaces 14a-14d of second monitor 140, but first monitor adapter 190 may further include electronics (e.g., signal processing circuitry, which may include processors, encoding engines, etc.) configured to process information (e.g., data derived from patient connector ports 222) for output from the multi-contact electrical interfaces 12a and 12b for display on second monitor 140.

For example, the signal processing circuitry of first monitor adapter 190 may encode or otherwise process data to generate video and audio signals so that they can be properly output by second monitor 140 according to its specifications, which are different from first monitor 120. By moving this signal processing circuitry from first monitor 120 to first monitor adapter 190, cost can be saved at first monitor 120. Furthermore, required processing power at first monitor 120 is reduced since the processing and/or generation of video, for example, may be offloaded from first monitor 120 to first monitor adapter 190. Furthermore, operating temperature of first monitor 120 may be reduced since the processing and/or generation of video, for example, is offloaded from first monitor 120 to first monitor adapter 190.

First monitor adapter 190 may include a visual electronic display 192 with similar capabilities to electronic visual display 126. When first monitor adapter 190 is connected to first monitor 120, the two displays 126 and 192 may join to form a single display area, thereby effectively expanding the display area of first monitor 120. The expanded display may be used when the expanded first monitor assembly 120, 190 is not docked to second monitor 140.

First monitor adapter 190 may also enable use of an auxiliary battery (not illustrated) that is inserted into first monitor 120. For example, first monitor 120 may be configured with a slot or cavity for receiving different sized auxiliary batteries and receiving power therefrom. A four-cell auxiliary battery may, for example, be inserted fully into the housing of first monitor 120. A larger auxiliary battery (e.g., a six-cell battery pack) may extend out of the housing of first monitor 120. Thus, the housing of first monitor adapter 190 may act as an extension of the housing of first monitor 120 to house the portion of the extended battery that extends from the housing of first monitor 120. When the end cap (i.e., the first monitor adapter 190) is removed from first monitor 120, the auxiliary battery is accessible so that it can be replaced, swapped out, and the like.

By placing the auxiliary battery in first monitor adapter 190, the workflow is significantly improved at the expense of size and weight to the expanded first monitor assembly. The workflow is improved since there is only one battery gauge and only one place to be concerned about charging batteries. First monitor 120 may be configured to transfer power from the battery or power received from monitor mount 160 to the components of first monitor adapter 190. First monitor adapter 190 may also be configured to transfer power to second monitor 140 via multi-contact electrical interfaces 12a and 12b.

First monitor 120 may be configured to transmit signals (e.g., data, control, and power signals) to first monitor adapter 190 via a connector 225 or connector port. Connector 225 may include 14 electrical contacts (e.g., pins or pin holes) matched with the 7-contact electrical interfaces 12a and 12b. First monitor adapter 190 may include a connector 191 (or connector port) that includes 14 electrical contacts (e.g., pin holes or pins) configured to electrically couple the electrical contacts of connector 225. Thus, the two connectors 225 and 191 may be configured to be mechanically and electrically coupled to each other when first monitor adapter 190 is detachably secured to first monitor 120. Connector 191 may be an internal flex cable with a video encoder chip to connect a PI board to the 14 pin connector 225. An FDP-Link III in the connector 225 and the connector 191 can be used to transfer video and audio signals. Thus, the contacts of connectors 225 and 191 may be provided as set forth in TABLE 1.

Thus, first monitor adapter 190 may be added to first monitor 120 to expand the display, add additional functionality, or enable an auxiliary battery to be connected and housed.

In addition, connector 225, implemented as a 14-contact connector or connector port in this example, may be connected directly to second monitor 140 when first monitor adapter 190 is not connected to first monitor 120. In this case, second monitor 140 may have a 14-contact mating connector that mechanically and electrically coupled to connector 225 of first monitor 120. In this way, connector 225 may be used in addition to or in the alternative to multi-contact electrical interfaces 12a and 12b shown in FIGS. 3A and 3B. Thus, the direct connection between connector 225 and the 14-contact mating connector of second monitor 140 may be used to transmit video, audio, power, and/or control signals from first monitor 120 to second monitor 140 as described above. An FDP-Link III in connector 225 and the 14-contact mating connector can be used to transfer video and audio signals.

While various embodiments have been disclosed, it will be apparent to those skilled in the art that various changes and modifications can be made which will achieve some of the advantages of the concepts disclosed herein without departing from the spirit and scope of the invention. It will be obvious to those reasonably skilled in the art that other components performing the same functions may be suitably substituted. It is to be understood that other embodiments may be utilized and structural or logical changes may be made without departing from the scope of the present invention. It should be mentioned that features explained with reference to a specific figure may be combined with features of other figures, even in those not explicitly mentioned. Such modifications to the general inventive concept are intended to be covered by the appended claims and their legal equivalents.

Furthermore, the following claims are hereby incorporated into the detailed description, where each claim may stand on its own as a separate example embodiment. While each claim may stand on its own as a separate example embodiment, it is to be noted that—although a dependent claim may refer in the claims to a specific combination with one or more other claims—other example embodiments may also include a combination of the dependent claim with the subject matter of each other dependent or independent claim. Such combinations are proposed herein unless it is stated that a specific combination is not intended. Furthermore, it is intended to include also features of a claim to any other independent claim even if this claim is not directly made dependent to the independent claim.

It is further to be noted that methods disclosed in the specification or in the claims may be implemented by a device having means for performing each of the respective acts of these methods. For example, the techniques described in this disclosure may be implemented, at least in part, in hardware, software, firmware, or any combination thereof. For example, various aspects of the described techniques may be implemented within one or more processors, including one or more microprocessors, central processing units (CPUs), digital signal processors (DSPs), application specific integrated circuits (ASICs), or any other equivalent integrated or discrete logic circuitry, as well as any combinations of such components and one or more machine learning algorithms.

Further, it is to be understood that the disclosure of multiple acts or functions disclosed in the specification or in the claims may not be construed as to be within the specific order. Therefore, the disclosure of multiple acts or functions will not limit these to a particular order unless such acts or functions are not interchangeable for technical reasons. Furthermore, in some embodiments a single act may include or may be broken into multiple sub acts. Such sub acts may be included and part of the disclosure of this single act unless explicitly excluded.

1. A monitor system, comprising:
a first monitor configured to receive physiological sensor data from at least one physiological sensor attached to a patient, the first monitor including:
a first electronic visual display,
a first multi-contact electrical interface, and
a second multi-contact electrical interface,
wherein the first and the second multi-contact electrical interfaces are configured to provide data signals derived from the physiological sensor data and power signals provided by a power source; and
a second monitor including:
a second electronic visual display,
a third multi-contact electrical interface configured to electrically couple with the first multi-contact electrical interface, and
a fourth multi-contact electrical interface configured to electrically couple with the second multi-contact electrical interface;
wherein the third and the fourth multi-contact electrical interfaces are configured to receive the data signals and the power signals from the first monitor, and wherein the second electronic visual display is configured to display images corresponding to the data signals.

2. The monitor system of claim 1, wherein:
the second monitor further includes:
a fifth multi-contact electrical interface configured to electrically couple with the first multi-contact electrical interface, and
a sixth multi-contact electrical interface configured to electrically couple with the second multi-contact electrical interface;
the fifth and the sixth multi-contact electrical interfaces are configured to receive the data signals and the power signals from the first monitor, and
the second electronic visual display is configured to display images corresponding to the data signal.

3. The monitor system of claim 2, wherein:
one of the third multi-contact electrical interface or the fifth multi-contact electrical interface is electrically coupled to the first multi-contact electrical interface at a time, and
one of the fourth multi-contact electrical interface or the sixth multi-contact electrical interface is electrically coupled to the second multi-contact electrical interface at a time.

4. The monitor system of claim 3, wherein:
the second monitor comprises a mounting surface with a mounting area configured to removably receive the first monitor therein,
the first electronic visual display oriented toward the mounting surface during a first mounting maneuver from a first lateral side of the mounting area, and
the first electronic visual display oriented toward the mounting surface during a second mounting maneuver from a second lateral side of the mounting area,
wherein:
the third multi-contact electrical interface and the fourth multi-contact electrical interface are coupled to the first and the second electrical interfaces, respectively, when the first monitor is mounted via the first mounting maneuver from the first lateral side of the mounting area, and
the fifth multi-contact electrical interface and the sixth multi-contact electrical interface are coupled to the first and the second electrical interfaces, respectively, when the first monitor is mounted via the second mounting maneuver from the second lateral side of the mounting area.

5. The monitor system of claim 2, wherein:
the first and the second multi-contact interfaces are arranged on opposite surfaces of the first monitor,
the third and the fourth multi-contact interfaces are arranged opposite to each other across the rear mounting area, and
the fifth and the sixth multi-contact interfaces are arranged opposite to each other across the rear mounting area.

6. A monitor assembly, comprising:
a first monitor configured to receive physiological sensor data from at least one physiological sensor attached to a patient, the first monitor including:
a housing,
a first electronic visual display, and
a first multi-contact electrical interface configured to transmit data signals derived from the physiological sensor data and power signals provided by a power source; and
a first monitor adapter configured to detachably couple to the first monitor to extend the housing of the first monitor, the first monitor adapter including a second multi-contact electrical interface configured to electrically couple to the first multi-contact electrical interface for receiving the data signals and the power signals therefrom.

7. The monitor assembly of claim 6, wherein the first monitor adapter comprises a second electronic visual display configured to be combined with the first electronic visual display when the first monitor adapter is coupled to the first monitor.

8. The monitor assembly of claim 6, wherein:
the first monitor is configured to receive a removable auxiliary battery having a portion that extends from an end of the housing, and
the first monitor adapter is an end cap enclosure that houses the portion of the removable auxiliary battery that extends from the end of the housing.

9. The monitor assembly of claim 6, further comprising:
a second monitor including a third electronic visual display,
wherein:
the first monitor adapter comprises a third multi-contact electrical interface and a fourth multi-contact electrical interface,
the third and the fourth multi-contact electrical interfaces are configured to provide the data signals and the power signals received by the second multi-contact electrical interface to the second monitor,
the second monitor includes a fifth multi-contact electrical interface configured to electrically couple with the third multi-contact electrical interface, and a sixth multi-contact electrical interface configured to electrically couple with the fourth multi-contact electrical interface;
the fifth and sixth multi-contact electrical interfaces are configured to receive the data signals and the power signals from the first monitor via the first monitor adapter; and
the second electronic visual display is configured to display images corresponding to the data signals.

10. The monitor assembly of claim 7, wherein the first monitor adapter comprises a video processor configured to generate the images based on the data signals, wherein the images are conformed to at least one parameter of the third electronic visual display.

11. A method of configuring monitor system, comprising:
providing a first monitor configured to receive physiological sensor data from at least one physiological sensor attached to a patient, the first monitor including:
a first electronic visual display,
a first multi-contact electrical interface, and
a second multi-contact electrical interface,
wherein the first and the second multi-contact electrical interfaces are configured to provide data signals derived from the physiological sensor data and power signals provided by a power source; and
providing a second monitor including:
a second electronic visual display,
a third multi-contact electrical interface configured to electrically couple with the first multi-contact electrical interface, and
a fourth multi-contact electrical interface configured to electrically couple with the second multi-contact electrical interface; and
arranging the third and the fourth multi-contact electrical interfaces to receive the data signals and the power signals from the first monitor,
wherein the second electronic visual display is configured to display images corresponding to the data signals.

12. The method of claim 11, further comprising:
further providing the second monitor with:
a fifth multi-contact electrical interface configured to electrically couple with the first multi-contact electrical interface, and
a sixth multi-contact electrical interface configured to electrically couple with the second multi-contact electrical interface; and
positioning the fifth and the sixth multi-contact electrical interfaces to receive the data signals and the power signals from the first monitor, the second electronic visual display being configured to display images corresponding to the data signal.

13. The method of claim 12, further comprising:
electrically coupling one of the third multi-contact electrical interface or the fifth multi-contact electrical interface to the first multi-contact electrical interface at a time, and
electrically coupling one of the fourth multi-contact electrical interface or the sixth multi-contact electrical interface to the second multi-contact electrical interface at a time.

14. The method of claim 13, further comprising:
providing the second monitor with a rear surface having a rear mounting area configured to removably receive the first monitor therein;
orienting the first electronic visual display toward the rear surface during a first mounting maneuver from a first lateral side of the rear mounting area and orienting the first electronic visual display toward the rear surface during a second mounting maneuver from a second lateral side of the rear mounting area;
arranging the third multi-contact electrical interface and the fourth multi-contact electrical interface to be coupled to the first and the second electrical interfaces, respectively, when the first monitor is mounted via the first mounting maneuver from the first lateral side of the rear mounting area; and
arranging the fifth multi-contact electrical interface and the sixth multi-contact electrical interface to be coupled to the first and the second electrical interfaces, respectively, when the first monitor is mounted via the second mounting maneuver from the second lateral side of the rear mounting area.

15. The method of claim 12, wherein:
arranging the first and the second multi-contact interfaces on opposite surfaces of the first monitor,
arranging the third and the fourth multi-contact interfaces opposite to each other across the rear mounting area, and
arranging the fifth and the sixth multi-contact interfaces opposite to each other across the rear mounting area.

16. A method of configuring a monitor assembly, comprising:
providing a first monitor configured to receive physiological sensor data from at least one physiological sensor attached to a patient, the first monitor including:
a housing,
a first electronic visual display, and
a first multi-contact electrical interface configured to transmit data signals derived from the physiological sensor data and power signals provided by a power source; and
providing a first monitor adapter configured to detachably couple to the first monitor to extend the housing of the first monitor, the first monitor adapter including a second multi-contact electrical interface configured to electrically couple to the first multi-contact electrical interface for receiving the data signals and the power signals therefrom.

17. The method of claim 16, further comprising:
providing the first monitor adapter with a second electronic visual display configured to be combined with the first electronic visual display when the first monitor adapter is coupled to the first monitor.

18. The method of claim 16 or 17, further comprising:
configuring the first monitor to receive a removable auxiliary battery having a portion that extends from an end of the housing; and
providing the first monitor adapter with an end cap enclosure that houses the portion of the removable auxiliary battery that extends from the end of the housing.

19. The method of claim 16, further comprising:
providing a second monitor including a third electronic visual display,
configuring a third multi-contact electrical interface and a fourth multi-contact electrical interface of the first monitor adaptor to provide the data signals and the power signals received by the second multi-contact electrical interface to the second monitor,
providing a fifth multi-contact electrical interface on the second monitor to electrically couple with the third multi-contact electrical interface;
providing sixth multi-contact electrical interface configured to electrically couple with the fourth multi-contact electrical interface;
configuring the fifth and sixth multi-contact electrical interfaces to receive the data signals and the power signals from the first monitor via the first monitor adapter; and
configuring the second electronic visual display to display images corresponding to the data signals.

20. The method of claim 17, further comprising:
providing the first monitor adapter with a video processor configured to generate the images based on the data signals, wherein the images are conformed to at least one parameter of the third electronic visual display.
